(19) United States
(12) Patent Application Publication
(10) Pub. No.: US 2020/0346212 A1
(43) Pub. Date: Nov. 5, 2020

(54) MICROFABRICATED DEVICE WITH HYDROPHILIC MICROWELLS AND HYDROPHOBIC INTERSTITIAL SPACE

(71) Applicant: General Automation Lab Technologies Inc., San Carlos, CA (US)

(72) Inventors: Alexander Hallock, San Carlos, CA (US); Marc Glazer, San Carlos, CA (US)

(21) Appl. No.: 16/865,202

(22) Filed: May 1, 2020

Related U.S. Application Data
(60) Provisional application No. 62/842,456, filed on May 2, 2019.

(51) Int. Cl. B01L 3/00; B01J 19/00; G01N 33/543

(52) U.S. Cl. CPC B01L 3/502715; B01J 19/0046; G01N 33/54366; B01L 3/502761; B01L 2300/0887; B01L 2300/044; B01L 2300/0829; B01L 2300/0874; B01J 2219/00659

(57) ABSTRACT

A method of modifying a microfabricated chip made of a hydrophobic material is provided. The microfabricated chip has a top surface including a plurality of microwells each having a bottom and a side wall, and interstitial space between the microwells. First, the ed chip is surface treated to render the surface of the bottom and side wall of the microwells and the interstitial space hydrophilic. Then, the surface of the interstitial space is treated to render it hydrophobic. A microfabricated chip having hydrophilic microwells and hydrophobic interstitial space is also provided.

### CROSS REFERENCE TO RELATED APPLICATION

This application claims the benefit of priority of U.S. Provisional Patent Application No. 62/842,456, filed May 2, 2019, the disclosure of which is incorporated by reference herein in its entirety.

### BACKGROUND

Microfluidic devices have been exploited in the drug discovery industry and for improving experimentation in a variety of areas in biology, such as cell culture. Advantages of micro analysis systems include reduced sample size, precise micro environmental control, and parallel operation in a single device yielding the ability to perform high throughput analyses.

Due to the microscale size of the fluid channels and reaction chambers on microfluidic devices, complex peripheral equipment is often required to manipulate the fluid flow on such devices. Capillary microfluidics can deliver liquids in a pre-programmed manner without peripheral equipment by exploiting capillary effects rendered by the surface chemistry of microchannels. However, precision lithography and channel surface treatment may be needed.

With platforms comprising a high-density array of microwells and with no microchannel interconnections, loading the array of wells and keeping the contents of the wells isolated from each other can be a challenge. In particular, if the platform is fabricated by a hydrophobic material, such as a hydrophobic plastic, loading aqueous solutions into the array of microwells may be impeded by surface tension and trapped air in the microwells.

### SUMMARY OF THE INVENTION

In one aspect, the present disclosure provides a method of modifying a microfabricated chip having a top surface including a plurality of microwells each having a bottom and a side wall, and interstitial space between the microwells, the microfabricated chip being made of a hydrophobic material. The method comprises, in the following order: (a) treating the microfabricated chip to render the surface of the bottom and side wall of the microwells and the interstitial space hydrophilic; and (b) selectively treating the surface of the interstitial space to render it hydrophobic.

In some embodiments of the method, step (a) comprises treating the microfabricated chip with plasma. In some embodiments of the method, step (a) comprises treating the microfabricated chip with one of corona discharge, ozone, and copper-enhanced oxidation.

In some embodiments of the method, step (a) comprises forming a hydrophilic layer of small molecule or polymer, e.g., by photochemical surface modification, on the surface of the bottom and side wall of the microwells and the interstitial space of the microfabricated chip.

In some embodiments of the method, step (b) comprises contacting an object with the surface of the interstitial space so as to impart hydrophobicity to the surface of the interstitial space.

In some embodiments, step (b) comprises selectively removing a top layer of the surface of the interstitial space.

In some embodiments, before step (b), the method further includes: (c) applying a hydrophilic liquid on the microfabricated device to fill at least a portion of each of the plurality of wells with the liquid. Such application of the hydrophilic liquid can cause a portion of the liquid to be retained on the interstitial space. In some of these embodiments, the method further comprises: after (c) and before (b): (d) removing the portion of liquid retained on the interstitial space. Removal of the restrained liquid can be accomplished by controlled evaporation, or by using a soft blade to swipe through the interstitial space surface, or by using an absorbent material to remove the retained liquid on the interstitial space by absorption.

In some embodiments, step (b) comprises spraying an organic solvent onto the surface of the interstitial space. In other embodiments, step (b) comprises forming a hydrophobic polymer layer on the surface of the interstitial space.

In another aspect, the present disclosure provides a method of modifying a microfabricated chip having a top surface including a plurality of microwells each having an interior surface and an interstitial space between the microwells, the microfabricated chip being made of a hydrophobic material. The method comprises, in the following order: (a) applying a hydrophilic liquid on the microfabricated chip so as to fill at least a portion of each of the plurality of wells with the hydrophilic liquid; (b) if any portion of the hydrophilic liquid remains on the interstitial space, removing the portion of the hydrophilic liquid from the interstitial space; and (c) converting the interior surface of the microwells to hydrophilic. The hydrophilic liquid can be an aqueous solution containing a water soluble polymer, such as poly(vinyl alcohol) (PVA).

In a further aspect, the present disclosure provides a microfabricated device having a top surface defining an array of microwells having a surface density of at least 750 microwells per cm², each microwell having a bottom and a side wall, and interstitial space between the microwells, the microfabricated device being made of a hydrophobic base material, where the internal surfaces of the microwells are modified to be hydrophilic and the interstitial space between the microwells is hydrophobic.

In a further aspect, the present disclosure provides a method of culturing and screening for at least one biological entity of interest using a microfabricated device having a top surface defining an array of microwells having a surface density of at least 750 microwells per cm², wherein each of the microwells has a bottom and a side wall and an interstitial space between the microwells, the microfabricated device being made of a hydrophobic material where the internal surfaces of the microwells are hydrophilic and the interstitial space between the microwells is hydrophobic. The method includes: loading a sample onto the microfabricated device such that at least one microwell of the array of microwells includes at least one cell and an amount of a nutrient; applying a membrane to the microfabricated device to retain the at least one cell and the nutrient in the at least one microwell of the array of microwells; without furnishing additional nutrient, culturing a plurality of cells from the at least one cell in the at least one microwell of the array of microwells; and analyzing the plurality of cells to determine a presence or absence of a biological entity of interest.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a perspective view illustrating a microfabricated device or chip in accordance with some embodiments.

FIGS. 2A-2C are top, side, and end views, respectively, illustrating dimensions of microfabricated device or chip in accordance with some embodiments.

FIGS. 3A and 3B are exploded and top views, respectively, illustrating a microfabricated device or chip in accordance with some embodiments.

FIG. 4 is a schematic depiction of a cross section of a microfabricated device in accordance with some embodiments, the microfabricated device including microwells where the internal surfaces of the microwells are hydrophilic and the interstitial space between the microwells is hydrophobic.

### DETAILED DESCRIPTION OF EMBODIMENTS OF THE INVENTION

An object of the present invention is to provide a microfabricated device (or chip) having a top surface defining an array of microwells and interstitial space between the microwells, the microfabricated device being made of a hydrophobic polymer material, where the internal surfaces of the microwells are hydrophilic and the interstitial space between the microwells is hydrophobic. The microwells each have a bottom and a side wall. The term “bottom” is used herein to indicate that the microwells have finite depth in the thickness direction of the microfabricated device and are not through holes across the microfabricated device. The bottom and the side wall may have clear boundaries between them, but can also be smoothly joined without obvious demarcation. Another object of the present invention is to provide methods to modify a microfabricated chip made of a hydrophobic material such that the internal surfaces of the microwells becomes hydrophilic and the interstitial space between the microwells remains hydrophobic. These characteristics would significantly simplify the loading, sealing, cell retention in the microwells, as well as downstream operations such as picking and transferring samples from microwells.

In some embodiments, the high density cell cultivation platform can be a microfabricated device (or a “chip”). As used herein, a microfabricated device or chip may define a high density array of microwells (or experimental units). For example, a microfabricated chip comprising a “high density” of microwells may include about 150 microwells per cm² to about 160,000 microwells or more per cm² (for example, at least 150 microwells per cm², at least 250 microwells per cm², at least 400 microwells per cm², at least 500 microwells per cm², at least 750 microwells per cm², at least 1,000 microwells per cm², at least 2,500 microwells per cm², at least 5,000 microwells per cm², at least 7,500 microwells per cm², at least 10,000 microwells per cm², at least 50,000 microwells per cm², at least 100,000 microwells per cm², or at least 160,000 microwells per cm²). A substrate of a microfabricated chip may include about or more than 10,000,000 microwells or locations. For example, an array of microwells may include at least 96 locations, at least 1,000 locations, at least 5,000 locations, at least 10,000 locations, at least 50,000 locations, at least 100,000 locations, at least 500,000 locations, at least 1,000,000 locations, at least 5,000,000 locations, or at least 10,000,000 locations. The arrays of microwells may form grid patterns, and be grouped into separate areas or sections. The dimensions of a microwell may range from nanoscopic (e.g., a diameter from about 1 to about 100 nanometers) to microscopic. For example, each microwell may have a diameter of about 1μm to about 800μm, a diameter of about 25μm to about 500μm, or a diameter of about 30μm to about 100μm. A microwell may have a diameter of about or less than 1μm, about or less than 5μm, about or less than 10μm, about or less than 25μm, about or less than 50μm, about or less than 100μm, about or less than 200μm, about or less than 30082 m, about or less than 400μm, about or less than 500μm, about or less than 600μm, about or less than 700μm, or about or less than 800μm. In exemplary embodiments, the diameter of the microwells can be about 100μm or smaller, or 50μm or smaller. A microwell may have a depth of about 25μm to about 100μm, e.g., about 1μm, about 5μm, about 10μm, about 25μm, about 50μm, about 100μm. It can also have greater depth, e.g., about 200μm, about 300μm, about 400μm, about 500μm. The spacing between adjacent microwells can range from about 25μm to about 500μm, or about 30μm to about 100μm.

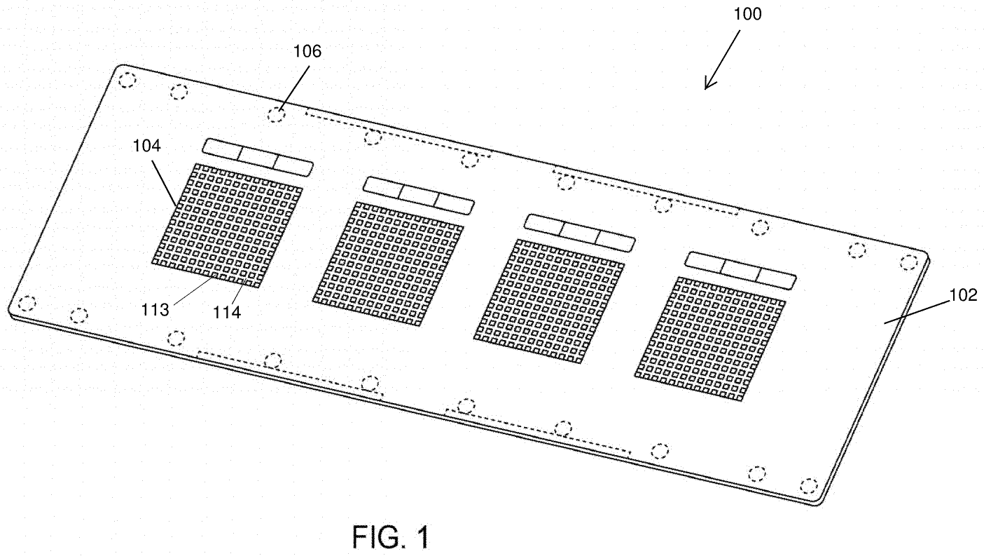

The microfabricated chip can have two major surfaces: a top surface and a bottom surface, where the microwells have openings at the top surface. Each microwell of the microwells may have an opening or cross section having any shape, e.g., round, hexagonal, square, or other shapes. Each microwell may include sidewalls. For microwells that are not round in their openings or cross sections, the diameter of the microwells described herein refer to the effective diameter of a circular shape having an equivalent area. For example, for a square shaped microwell having side lengths of 10×10 microns, a circle having an equivalent area (100 square microns) has a diameter of 11.3 microns. Each microwell may include a sidewall or sidewalls. The sidewalls may have a cross-sectional profile that is straight, oblique, and/or curved. Each microwell includes a bottom which can be flat, round, or of other shapes. The microfabricated chip (with the microwells thereon) may be manufactured from a polymer, e.g., a cyclic olefin polymer, via precision injection molding or some other process such as embossing. Other material of construction is also available, such as silicon and glass. The chip may have a substantially planar major surface. FIG. 1 shows a schematic depiction of a microfabricated chip, whose edges are generally parallel to the directions of the rows and the columns of the microwells on the chip.

The high density microwells on the microfabricated chip can be used for receiving a sample comprising at least one biological entity (e.g., at least one cell). The term “biological entity” may include, but is not limited to, an organism, a cell, a cell component, a cell product, and a virus, and the term “species” may be used to describe a unit of classification, including, but not limited to, an operational taxonomic unit (OTU), a genotype, a phylotype, a phenotype, an ecotype, a history, a behavior or interaction, a product, a variant, and an evolutionarily significant unit. The high density microwells on the microfabricated chip can be used to conduct various experiments, such as growth or cultivation or screening of various species of bacteria and other microorganisms (or microbes) such as aerobic, anaerobic, and/or facultative aerobic microorganisms. The microwells may be used to conduct experiments with eukaryotic cells such as mammalian cells. Also, the microwells can be used to conduct various genomic or proteomic experiments, and may contain cell products or components, or other chemical or biological substances or entities, such as a cell surface (e.g., a cell membrane or wall), a metabolite, a vitamin, a hormone, a neurotransmitter, an antibody, an amino acid, an enzyme, a protein, a saccharide, ATP, a lipid, a nucleoside, a nucleotide, a nucleic acid (e.g., DNA or RNA), a chemical, e.g., a dye, enzyme substrate, etc.

In some embodiments, the high density cell cultivation platform can be droplet based, e.g., instead of array(s) of wells as experimental units on a microfabricated chip, a population of discrete droplets can be used to retain cells, media and other components for cell cultivation. Droplet generation methods, especially when combined with cell-sorter-on-a-chip type instrumentation, may be used to grow and screen microbes from a complex environmental sample. Droplets may be produced at several hundred Hz, meaning millions of drops can be produced in a few hours. A simple chip-based device may be used to generate droplets and the droplets may be engineered to contain a single cell. A system for generating droplets containing cell suspensions may contain one or small numbers of cells. The droplets can be emulsions, double emulsion, hydrogel, bubbles and complex particles, etc. For example, aqueous drops may be suspended in a nonmiscible liquid keeping them apart from each other and from touching or contaminating any surfaces. The volume of a droplet can be somewhere between 10 fl and 1μL, and highly monodisperse droplets can be made from a few nanometers up to 500μm in diameter. A droplet-based microfluidic system may be used to generate, manipulate, and/or incubate small droplets. Cell survival and proliferation can be similar to control experiments in bulk solution. Fluorescence screening of droplets may be done on-chip and at a rate of, for example, 500 drops per second. Droplets may be merged to create a new droplet or a reagent added to a droplet. Droplets can be passed in a microchannel in a single file and interrogated by a spectroscopic method, e.g., using a fluorescence detector to detect fluorescence emitted from the droplets, and those droplets that are determined to meet certain criteria (e.g., emitting fluorescence at certain wavelength) can be selected via diversion into a branched channel from which the droplet can be pooled or harvested. The diversion or switching of flow can be accomplished by valves, pump, applying an external electric field, etc.

In various embodiments, a cell may be Archaea, Bacteria, or Eukaryota (e.g., fungi). For example, a cell may be a microorganism, such as an aerobic, anaerobic, or facultative aerobic microorganisms. A virus may be a bacteriophage. Other cell components/products may include, but are not limited to, proteins, amino acids, enzymes, saccharides, adenosine triphosphate (ATP), lipids, nucleic acids (e.g., DNA and RNA), nucleosides, nucleotides, cell membranes/walls, flagella, fimbriae, organelles, metabolites, vitamins, hormones, neurotransmitters, and antibodies.

For the cultivation of cells, a nutrient is often provided. A nutrient may be defined (e.g., a chemically defined or synthetic medium) or undefined (e.g., a basal or complex medium). A nutrient may include or be a component of a laboratory-formulated and/or a commercially manufactured medium (e.g., a mix of two or more chemicals). A nutrient may include or be a component of a liquid nutrient medium (i.e., a nutrient broth), such as a marine broth, a lysogeny broth (e.g., Luria broth), etc. A nutrient may include or be a component of a liquid medium mixed with agar to form a solid medium and/or a commercially available manufactured agar plate, such as blood agar.

A nutrient may include or be a component of selective media. For example, selective media may be used for the growth of only certain biological entities or only biological entities with certain properties (e.g., antibiotic resistance or synthesis of a certain metabolite). A nutrient may include or be a component of differential media to distinguish one type of biological entity from another type of biological entity or other types of biological entities by using biochemical characteristics in the presence of specific indicator (e.g., neutral red, phenol red, eosin y, or methylene blue).

A nutrient may include or be a component of an extract of or media derived from a natural environment. For example, a nutrient may be derived from an environment natural to a particular type of biological entity, a different environment, or a plurality of environments. The environment may include, but is not limited to, one or more of a biological tissue (e.g., connective, muscle, nervous, epithelial, plant epidermis, vascular, ground, etc.), a biological fluid or other biological product (e.g., amniotic fluid, bile, blood, cerebrospinal fluid, cerumen, exudate, fecal matter, gastric fluid, interstitial fluid, intracellular fluid, lymphatic fluid, milk, mucus, rumen content, saliva, sebum, semen, sweat, urine, vaginal secretion, vomit, etc.), a microbial suspension, air (including, e.g., different gas contents), supercritical carbon dioxide, soil (including, e.g., minerals, organic matter, gases, liquids, organisms, etc.), sediment (e.g., agricultural, marine, etc.), living organic matter (e.g., plants, insects, other small organisms and microorganisms), dead organic matter, forage (e.g., grasses, legumes, silage, crop residue, etc.), a mineral, oil or oil products (e.g., animal, vegetable, petrochemical), water (e.g., naturally-sourced freshwater, drinking water, seawater, etc.), and/or sewage (e.g., sanitary, commercial, industrial, and/or agricultural wastewater and surface runoff).

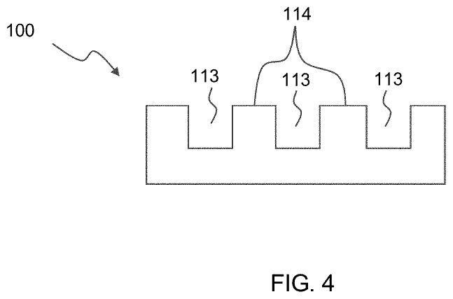

FIG. 1 is a perspective view illustrating a microfabricated device or chip in accordance with some embodiments. Chip 100 includes a substrate shaped in a microscope slide format with injection-molded features on top surface 102. The features include four separate microwell arrays (or microarrays) 104 as well as ejector marks 106. The microwells 113 (spaced by interstitial space 114) in each microarray are arranged in a grid pattern with well-free margins around the edges of chip 100 and between microarrays 104. FIG. 4 shows a schematic cross section view of a portion of a microfabricated device or chip of the present disclosure, where the internal surfaces (side wall and bottom) of the microwells 113 are hydrophilic and the interstitial space 114 between the microwells is hydrophobic.

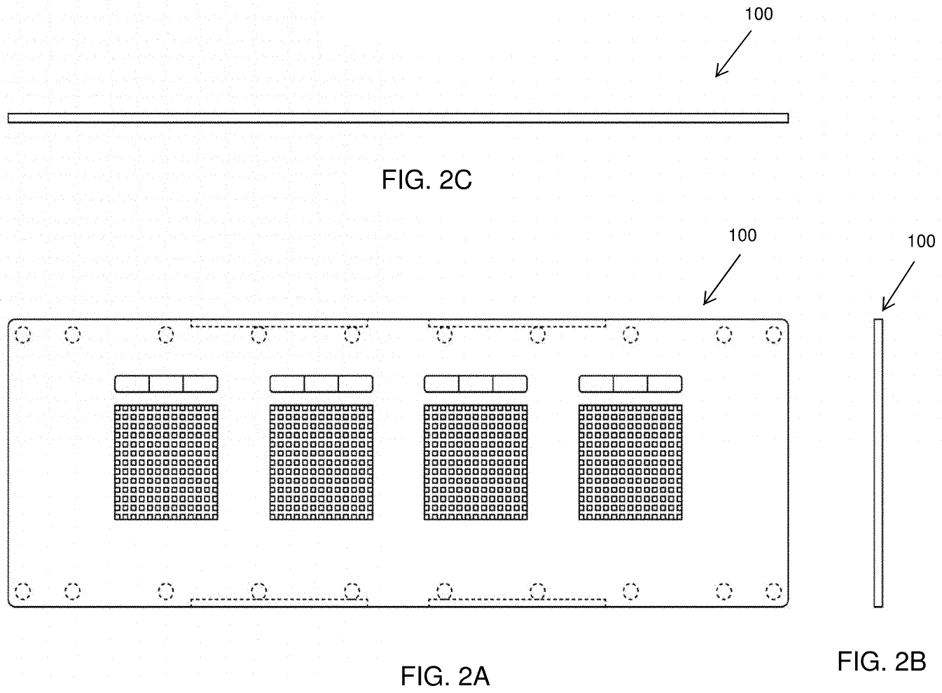

FIGS. 2A-2C are top, side, and end views, respectively, illustrating dimensions of chip 100 in accordance with some embodiments. In FIG. 2A, the top of chip 100 is approximately 25.5 mm by 75.5 mm. In FIG. 2B, the end of chip 100 is approximately 25.5 mm by 0.8 mm. In FIG. 2C, the side of chip 100 is approximately 75.5 mm by 0.8 mm.

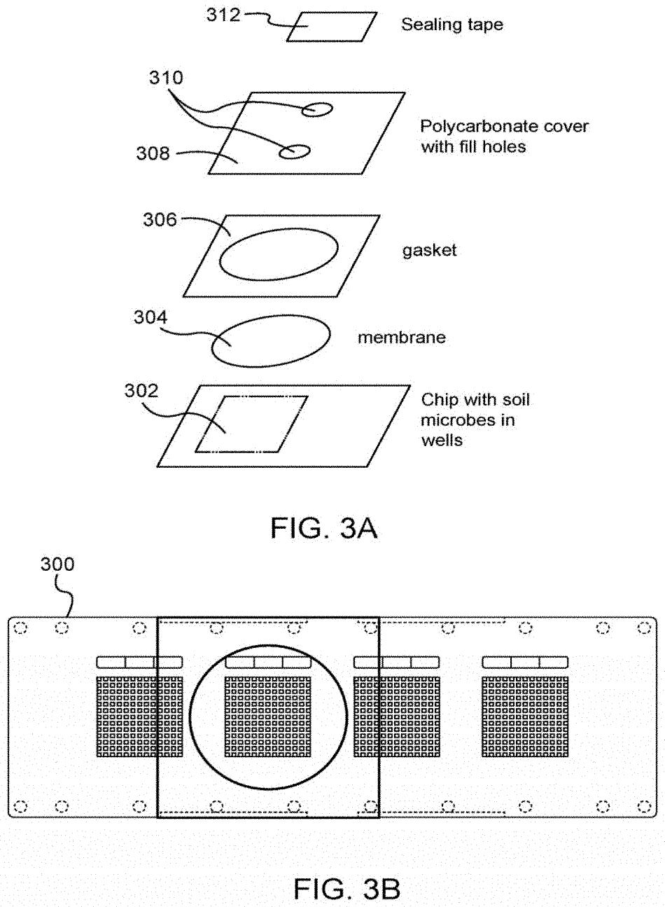

After a sample is loaded on a microfabricated device, a membrane may be applied to at least a portion of a microfabricated device. FIG. 3A is an exploded diagram of the microfabricated device 300 shown from a top view in FIG. 3B in accordance with some embodiments. Device 300 includes a chip with an array of wells 302 holding, for example, soil microbes. A membrane 304 is placed on top of the array of wells 302. A gasket 306 is placed on top of the membrane 304. A cover 308 with fill holes 310 is placed on top of the gasket 306. Finally, sealing tape 312 is applied to the cover 308.

A membrane may cover at least a portion of a microfabricated device including one or more experimental units or microwells. For example, after a sample is loaded on a microfabricated device, at least one membrane may be applied to at least one microwell of a high density array of microwells. A plurality of membranes may be applied to a plurality of portions of a microfabricated device. For example, separate membranes may be applied to separate subsections of a high density array of microwells.

A membrane may be connected, attached, partially attached, affixed, sealed, and/or partially sealed to a microfabricated device to retain at least one biological entity in the at least one microwell of the high density array of microwells. For example, a membrane may be reversibly affixed to a microfabricated device using lamination. A membrane may be punctured, peeled back, detached, partially detached, removed, and/or partially removed to access at least one biological entity in the at least one microwell of the high density array of microwells.

A portion of the population of cells in at least one experimental unit, well, or microwell may attach to a membrane (via, e.g., adsorption). If so, the population of cells in at least one experimental unit, well, or microwell may be sampled by peeling back the membrane such that the portion of the population of cells in the at least one experimental unit, well, or microwell remains attached to the membrane.

A membrane may be impermeable, semi-permeable, selectively permeable, differentially permeable, and/or partially permeable to allow diffusion of at least one nutrient into the at least one microwell of a high density array of microwells. For example, a membrane may include a natural material and/or a synthetic material. A membrane may include a hydrogel layer and/or filter paper. In some embodiments, a membrane is selected with a pore size small enough to retain at least some or all of the cells in a microwell. For mammalian cells, the pore size may be a few microns and still retain the cells. However, in some embodiments, the pore size may be less than or equal to about 0.2μm, such as 0.1μm. An impermeable membrane has a pore size approaching zero. It is understood that the membrane may have a complex structure that may or may not have defined pore sizes.

In one aspect, the present invention provides a method of modifying a microfabricated chip as described herein. The chip is made of a hydrophobic material, such as a plastic. The chip has a top surface including a plurality of microwells each having a bottom and a side wall, and interstitial space between the microwells on the top surface. The method includes first treating the microfabricated chip to render the surface of the bottom and side wall of the microwells and the interstitial space hydrophilic (the hydrophilic treatment step); and then selectively treating the surface of the interstitial space to render it hydrophobic (the hydrophobic treatment step).

With an untreated chip made of a hydrophobic polymer material, aqueous samples may not simply enter the microwells. Instead, liquid can sit on the interstitial space between the microwells. With the interior surface of the microwells rendered hydrophilic while retaining the hydrophobicity of the interstitial space, loading liquid samples into the microwells can be made easier.

To render the overall top surface of the microfabricated chip (including the well interior space and the interstitial space) hydrophilic, the microfabricated chip can be treated by plasma in the presence of a gas containing oxygen (air or pure oxygen), e.g., at powers of 30 W and higher, for treatment times of 1 minute and longer. Such treatments create hydrophilic functional groups on polymers including carboxylic acids, aldehydes, amines, and others, depending upon the particular polymer composition and plasma treatment. Alternatively, the microfabricated chip can undergo an ozone treatment (e.g., 1 L/min, 25 minutes with stage at 60 □), UV/ozone (UVO) treatment, corona discharge, or copper enhanced oxidation. In some embodiments, a thin layer of a metal oxide can be deposited across the chip. Examples are titanium or aluminum oxide, which can be readily deposited by several methods, including physical deposition (sputtering).

In some embodiments, the hydrophilic treatment step comprises forming a hydrophilic layer of small molecule or polymer on the surface of the bottom and side wall of the microwells and the interstitial space of the microfabricated chip. For example, plasma enhanced chemical vapor deposition and/or photochemical surface modification can be used. Such small molecule or polymer layer can be formed on top of the fresh “active” surface treated by the plasma, ozone or other treatments. For example, cyclic olefin copolymer (COC) surface may be modified by copper catalyzed peroxidative oxidation to introduce surface hydroxyl groups (which may be further modified to form a hybrid surface). See Carvalho et al., ACS Applied Materials and Interfaces, 2017, 9, 16644. As another example, to increase hydrophilicity of COC surfaces, poly(ethylene glycol) methacrylate (PEGMA) can be photografted using a two-step sequential approach which includes forming covalently-bound surface initiators and then graft polymerization of PEGMA from these initiators. See Stachowiak et al., J. Sep. Sci. 2007, 30, 1088.

In some embodiments, the hydrophobicity treatment step comprises contacting an object with the surface of the interstitial space so as to impart hydrophobicity to the surface of the interstitial space. The object can comprise a substrate of a PDMS stamp. The PDMS stamp may include a planar surface (for contacting the interstitial space of the microfabricated chip) with remnant unpolymerized dimethylsiloxane, or other silane molecules (such as octadecyltrimethoxysilane (ODTMS)). Upon contact with the top surface of the microfabricated chip (but not the interior surface of the microwells), the unpolymerized dimethylsiloxane or other silanes can react to the hydroxy or carboxyl groups on the activated surface of the interstitial space, resulting in a formation of a hydrophobic silane layer formed on top of the interstitial space. In one embodiment, a membrane containing a hydrophobic silicone adhesive can be used to apply to the top surface of a microfabricated chip and then peeled off, leaving behind a thin layer of residual polymerized and/or unpolymerized PDMS on the interstitial space between the microwells.

In case a thin layer of a metal oxide has been previously deposited on the chip (in wells and interstitial space), a PDMS stamp can then be used to transfer a hydrophobic phosphonic acid, such as octadecylphosphonic acid (ODPA), to the interstitial space. Phosphonic acids have been shown to bind strongly and selectively to aluminum and titanium oxides.

In some embodiments, the hydrophobicity treatment step can also be accomplished by selectively removing a top layer of the surface of the interstitial space. For example, an organic solvent such as toluene can be used to partially etch away a top thin layer on the interstitial space. The solvent can be impregnated into a PDMS stamp and the extent of the etching can be controlled by the amount of the solvent impregnated as well as the contact time between the PDMS stamp and the microfabricated chip.

In other embodiments, a thin layer from the interstitials can be subtracted and such that the interstitials can be converted back to the underlying hydrophobic substrate is to use chemical mechanical polishing, which is a hybrid of chemical etching and free abrasive polishing. The process uses an abrasive and corrosive chemical slurry in conjunction with a very flat polishing pad which can rotate a high speed. The flat face of the polishing pad can be held with pressure against the top surface of the microfabricated pad and with the help of the corrosive chemical, wear off desired depth of material off the interstitial space of the microfabricated chip.

In some embodiments, before the hydrophobicity treatment step, the microwells of the microfabricated chip can be first filled with a hydrophilic liquid on the microfabricated device so as to protect the interior surface of the microwells from further treatment. This microwell filling step can be done using a glass slide spreading a reservoir of liquid on the top surface of the microfabricated chip. This step may introduce some liquid retained on top of the interstitial space. Such unwanted liquid can be removed by using a soft blade to swipe through the interstitial space surface. The material for the soft blade needs to be compliant enough to adhere to any surface topology of the surface, hydrophilic enough to attract and push liquid off of the interstitials, but not so hydrophilic so as to absorb all of the liquid in the wells.

Alternatively or in addition, the unwanted liquid can be removed by an absorbent material. The material needs to be compliant enough to adhere to any surface topology of the surface, and have sufficient adsorption capacity to remove liquid from the interstitials. But it must not be so absorbent as to remove all of the liquid in the wells.

Further alternatively or in addition, unwanted liquid sitting on the interstitial space can be removed by controlled evaporation, i.e., by providing sealed environment around the microfabricated chip with controlled humidity such that the interstitials is dried but sufficient amount of liquid is still retained in the microwells.

Once the microwells are protected with liquid, methods for transferring hydrophobic materials other than directly contacting the microfabricated chip with a stamp can be used to impart hydrophobicity on the interstitial space of the microfabricated chip. In a sense, the liquid-filled microwells serve as a mask for the microwells. For example, an organic solvent can be sprayed onto the surface of the microfabricated chip to etch back the hydrophilic layer previously formed on the interstitial space. Solutions or suspensions comprising silanes can also be sprayed on the top surface of the microfabricated chip to form a hydrophobic film on the interstitial space.

Other polymers and small molecules may also be sprayed, printed, or lithographically patterned onto the interstitial space by utilizing “grafting from” or “grafting to” techniques to bury the previously hydrophilic surface with hydrophobic film. CVD or iCVD (initiated chemical vapor deposition) can also be used to deposit polymeric thin films on top of the interstitial space. After the interstitial space is treated, the liquid in the microwells can be removed.

In another approach, the initial hydrophobicity treatment step for the overall top surface of the microfabricated chip can be omitted. Instead, a hydrophilic liquid is first applied on the microfabricated chip so as to fill at least a portion of each of the plurality of wells with the hydrophilic liquid. If there is any portion of the hydrophilic liquid remaining on the interstitial space, such unwanted hydrophilic liquid is removed from the interstitial space (e.g., using the methods described above). Finally, the interior surface of the microwells are converted hydrophilic. The hydrophilic liquid to fill the microwells can include a surfactant at appropriate concentration, such that the polar tail group of the surfactant will adsorb to the hydrophobic well surface, and the hydrophilic head group will thereby give the surface of the well hydrophilicity. The liquid can be evaporated away leaving the surfactant onto the interior well surface. The concentration of the surfactant is low enough to ensure that only in the wells available surfactant can form a continuous coating, but residual on the interstitials will not have a substantial effect. In other embodiments, the hydrophilic liquid can include a soluble polymer, such as polyvinyl alcohol. When water is dried out, polyvinyl alcohol can form a thin film covering the interior surface of the microwells. The amount of polyvinyl alcohol left on the interstitials is not enough to form a continuous film, and therefore does not fundamentally change the hydrophobic nature of the interstitial space. In yet other embodiments, the hydrophilic liquid can include polymerizable compounds, such as acrylic acids, and polymerization initiators that can be activated by irradiation or other conditions. The polymerization can be initiated and carried out in the microwells to form a thin hydrophilic film on the interior surface of the microwells.

In alternative embodiments, a thin film mask can be placed on the top surface of the microfabricated chip, the mask having through holes matching the dimensions and relative locations of the microwells of the microfabricated chip, such that the interstitial space on the microfabricated chip is covered by the mask while the microwells are exposed. Thereafter, the exposed microwells can be treated by an oxygen plasma, lithography, spray coating of a hydrophilic material, and other techniques described herein to render the interior surface of the microwells hydrophilic. Then the thin film mask is removed, resulting in a microfabricated chip with hydrophilic microwells and hydrophobic interstitial space.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of modifying a microfabricated chip having a top surface including a plurality of microwells each having a bottom and a side wall, and interstitial space between the microwells, the microfabricated chip being made of a hydrophobic material, the method comprising, in the following order:
(a) treating the microfabricated chip to render the surface of the bottom and side wall of the microwells and the interstitial space hydrophilic; and
(b) selectively treating the surface of the interstitial space to render it hydrophobic.

2. The method of claim 1, wherein (a) comprises treating the microfabricated chip with plasma.

3. The method of claim 1, wherein (a) comprises treating the microfabricated chip with one of corona discharge, ozone, and copper-enhanced oxidation.

4. The method of claim 1, wherein (a) comprises forming a hydrophilic layer of small molecule or polymer on the surface of the bottom and side wall of the microwells and the interstitial space of the microfabricated chip.

5. The method of claim 4, wherein (a) comprises photochemical surface modification.

6. The method of claim 1, wherein (b) comprises contacting an object with the surface of the interstitial space so as to impart hydrophobicity to the surface of the interstitial space.

7. The method of claim 1, wherein (b) comprises selectively removing a top layer of the surface of the interstitial space.

8. The method of claim 1, further comprising, before (b):
(c) applying a hydrophilic liquid on the microfabricated device to fill at least a portion of each of the plurality of wells with the liquid.

9. The method of claim 8, wherein (c) causes a portion of the liquid to be retained on the interstitial space, the method further comprising, after (c) and before (b):
(d) removing the portion of liquid retained on the interstitial space.

10. The method of claim 9, wherein (d) comprises controlled evaporation.

11. The method of claim 9, wherein (d) comprises using a soft blade to swipe through the interstitial space surface.

12. The method of claim 10, wherein (d) comprises using an absorbent material to remove the retained liquid on the interstitial space by absorption.

13. The method of claim 9, wherein (b) comprising spraying an organic solvent onto the surface of the interstitial space.

14. The method of claim 9, wherein (b) comprising forming a hydrophobic polymer layer on the surface of the interstitial space.

15. A method of modifying a microfabricated chip having a top surface including a plurality of microwells each having an interior surface and an interstitial space between the microwells, the microfabricated chip being made of a hydrophobic material, the method comprising, in such order:
(a) applying a hydrophilic liquid on the microfabricated chip so as to fill at least a portion of each of the plurality of wells with the hydrophilic liquid;
(b) if any portion of the hydrophilic liquid remains on the interstitial space, removing the portion of the hydrophilic liquid from the interstitial space; and
(c) converting the interior surface of the microwells to hydrophilic.

16. The method of claim 15, wherein the hydrophilic liquid is an aqueous solution comprising a water soluble polymer.

17. The method of claim 16, wherein the water soluble polymer comprises poly(vinyl alcohol) (PVA).

18. (canceled)

19. A method of culturing and screening for at least one biological entity of interest using a microfabricated device having a top surface defining an array of microwells having a surface density of at least 750 microwells per cm², wherein each of the microwells has a bottom and a side wall and an interstitial space between the microwells, the microfabricated device being made of a hydrophobic material where the internal surfaces of the microwells are hydrophilic and the interstitial space between the microwells is hydrophobic, the method comprising:
loading a sample onto the microfabricated device such that at least one microwell of the array of microwells includes at least one cell and an amount of a nutrient;
applying a membrane to the microfabricated device to retain the at least one cell and the nutrient in the at least one microwell of the array of microwells;
without furnishing additional nutrient, culturing a plurality of cells from the at least one cell in the at least one microwell of the array of microwells; and
analyzing the plurality of cells to determine a presence or absence of a biological entity of interest.
